(19) United States
(12) Patent Application Publication
(10) Pub. No.: US 2010/0121188 A1
(43) Pub. Date: May 13, 2010

(54) REDUCING CONTRAST AGENT-INDUCED TOXICITY

(76) Inventors: Gurpreet S. Sandhu, Rochester, MN (US); David R. Holmes, III, Rochester, MN (US); David R. Holmes, JR., Rochester, MN (US); Nicole P. Sandhu, Rochester, MN (US); Robert D. Simari, Rochester, MN (US)

Correspondence Address: FISH & RICHARDSON P.C., PO BOX 1022, MINNEAPOLIS, MN, 55440-1022, US

(21) Appl. No.: 12/445,062

(22) PCT Filed: Oct. 10, 2007
(86) PCT No.: PCT/US07/80979

§ 371 Date: Jan. 11, 2010

Related U.S. Application Data
(60) Provisional application No. 60/850,836, filed on Oct. 10, 2006.

(51) Int. Cl. A61B 6/10; A61M 25/00; A61M 25/10

(52) U.S. Cl. 600/435; 604/523; 604/96.01; 604/508

(57) ABSTRACT

This document provides methods and materials that can be used to reduce or prevent contrast agent-induced toxicity. For example, devices for capturing contrast agents from blood are provided.

### CROSS REFERENCE RELATED APPLICATIONS

This document claims priority to U.S. Provisional Application Ser. No. 60/850,836, filed on Oct. 10, 2006, the contents of which are herein incorporated by reference in their entirety.

### BACKGROUND

1. Technical Field

This document provides methods and materials related to reducing or preventing a contrast agent-induced toxicity (e.g., a contrast agent-induced nephropathy).

2. Background Information

Nephropathy induced by contrast agents is a complication of diagnostic and therapeutic procedures that require administering a contrast agent to patients. In fact, contrast agent-induced nephropathy is the third leading cause of hospital-acquired acute renal failure. Contrast agent-induced toxicity can result in the need for dialysis, prolonged hospitalization, and an increased risk of death.

### SUMMARY

This document provides methods and materials that can be used to reduce or prevent contrast agent-induced toxicity. For example, this document provides methods that can include administering a contrast agent that contains a material capable of being attracted to a magnetic field. Such a contrast agent can be administered to an organ to be imaged, a body part to be imaged, or the blood supply upstream of the organ or body parts to be imaged. In some cases, a capture element capable of supplying a magnetic field can be positioned in the blood stream downstream of the organ or body parts to be imaged such that a percentage of the administered contrast agent containing a material capable of being attracted to a magnetic field is captured before proceeding to other locations of the body. This document also provides contrast agents, capture elements, kits including contrast agents and capture elements, and methods for using such contrast agents and capture elements to reduce or prevent contrast agent-induced toxicity. The methods and materials provided herein can be used in a minimally invasive manner to reduce a patient's risk of experiencing contrast agent-induced toxicity such as contrast agent-induced nephropathy.

In general, one aspect of this documents features a device comprising, or consisting essentially of: (a) a catheter configured to be inserted into a blood vessel of a mammal; and (b) a capture element comprising: (i) an elongate member, and (ii) a magnetic field source, wherein the capture element is configured to be at least partially housed within the guide catheter, and wherein the magnetic field source is movable, relative to the guide catheter. The elongate member can comprise a proximal end portion and a distal end portion, and wherein the magnetic field source can be located at or near the distal end portion. The magnetic field source can be movable, relative to the guide catheter, in a direction away from a distal end of the guide catheter when the distal end of the guide catheter is located within the circulatory system of the mammal. The mammal can be a human. The guide catheter can be between 10 cm and 125 cm in length. The guide catheter can be between 0.1 mm and 0.4 cm in diameter (e.g., between 0.1 and 2.5 cm). The elongate member can comprise a wire. The magnetic field source can comprise an electromagnet. The capture element can comprise a coating located at least partially around the magnetic field source. The coating can comprise an inner surface and an outer surface, wherein the outer surface is configured to contact a contrast agent containing a material capable of being attracted to a magnetic field when a distal end of the guide catheter is located within the circulatory system of the mammal and the mammal received the contrast agent. The coating can comprise projections that extend away from the magnetic field source, thereby increasing the surface area of the coating for contact with the contrast agent. The coating can be a pleated coating. The capture element can comprise a fiber network located at or near a distal end portion of the elongate member, wherein the fiber network comprises the magnetic field source. The fiber network can define spaces having a diameter capable of allowing blood flow through the fiber network. The capture element can comprise a mesh located at or near a distal end portion of the elongate member, wherein the mesh comprises the magnetic field source. The mesh can define spaces having a diameter capable of allowing blood flow through the mesh. The mesh can be an expandable mesh. The capture element can comprise an expandable balloon attached to an expandable mesh.

In another aspect, this documents features a method for reducing the amount of circulating contrast agent within a mammal, wherein the mammal received a contrast agent containing a material capable of being attracted to a magnetic field. The method comprises, or consists essentially of: (a) obtaining a device comprising: (i) a catheter configured to be inserted into a blood vessel of a mammal; and (ii) a capture element comprising: (1) an elongate member, and (2) a magnetic field source, wherein the capture element is configured to be at least partially housed within the guide catheter, and wherein the magnetic field source is movable, relative to the guide catheter, (b) inserting the magnetic field source into the circulatory system, and (c) exposing blood of the mammal to a magnetic field from the magnetic field source under conditions wherein the contrast agent is attracted to the magnetic field and contacts the device, thereby reducing the amount of circulating contrast agent within the mammal.

In another aspect, this documents features a method for performing a contrast agent imaging procedure having reduced risk of contrast agent-induced toxicity. The method comprises, or consists essentially of: (a) administering a contrast agent into a mammal, wherein the contrast agent comprises a material capable of being attracted to a magnetic field, (b) obtaining a device comprising: (i) a catheter configured to be inserted into a blood vessel of a mammal; and (ii) a capture element comprising: (1) an elongate member, and (2) a magnetic field source, wherein the capture element is configured to be at least partially housed within the guide catheter, and wherein the magnetic field source is movable, relative to the guide catheter, (c) obtaining an image from the mammal, (d) inserting the magnetic field source into the circulatory system, and (e) exposing blood of the mammal to a magnetic field from the magnetic field source under conditions wherein the contrast agent is attracted to the magnetic field and contacts the device, thereby reducing the amount of circulating contrast agent within the mammal.

In another aspect, this documents features a device comprising, or consisting essentially of: (a) a guide catheter defining a lumen and having a proximal end and a distal end, wherein the guide catheter is configured to be inserted into a blood vessel of a mammal; and (b) a capture element comprising: (i) an elongate member having a proximal end portion and a distal end portion, and (ii) a magnetic field source located at or near the distal end portion, wherein the capture element is configured to be at least partially housed within the guide catheter, and wherein the magnetic field source is movable, relative to the guide catheter, in a direction away from the distal end of the guide catheter when the distal end of the guide catheter is located within the circulatory system of the mammal. The mammal can be a human. The guide catheter can be between 10 cm and 125 cm in length. The guide catheter can be between 100 cm and 110 cm in length. The guide catheter can be between 0.1 mm and 0.4 cm in diameter (e.g., between 0.1 and 2.5 cm in diameter). The guide catheter can be between 0.2 cm and 0.3 cm in diameter. The elongate member can comprise a wire. The magnetic field source can comprise a permanent magnet. The magnetic field source can comprise an electromagnet. The capture element can comprise a coating located at least partially around the magnetic field source. The coating can comprise an inner surface and an outer surface, wherein the outer surface is configured to contact a contrast agent containing a material capable of being attracted to a magnetic field when the distal end of the guide catheter is located within the circulatory system of the mammal and the mammal received the contrast agent. The coating can comprise projections that extend away from the magnetic field source, thereby increasing the surface area of the coating for contact with the contrast agent. The coating can be a pleated coating. The capture element can comprise a coating surrounding the magnetic field source. The capture element can comprise a fiber network located at or near the distal end portion, wherein the fiber network comprises the magnetic field source. The fiber network can define spaces having a diameter capable of allowing blood flow through the fiber network. The fiber network can define spaces having a diameter between about 5 nm and 1 cm. The capture element can comprise a mesh located at or near the distal end portion, wherein the mesh comprises the magnetic field source. The mesh can define spaces having a diameter capable of allowing blood flow through the mesh. The mesh can define spaces having a diameter between about 5 nm and 1 cm. The mesh can be an expandable mesh. The mesh can comprise nitinol. The capture element can comprise an expandable balloon attached to an expandable mesh.

In another aspect, this documents features a method for reducing the amount of circulating agent within a mammal, wherein the mammal received an agent containing a material capable of being attracted to a magnetic field. The method comprises, or consists essentially of: (a) inserting a device comprising a magnetic field source into a blood vessel of the mammal, and (b) exposing blood of the mammal to a magnetic field from the magnetic field source under conditions wherein the agent is attracted to the magnetic field and contacts the device, thereby reducing the amount of circulating agent within said mammal. The mammal can be a human. The magnetic field source can be inserted into a right atrium, superior vena cava, inferior vena cava, coronary sinus, or pulmonary artery. The magnetic field can have a Gaussian strength of between 0.1 and 10,000 Gauss. The agent can be a contrast agent.

In another aspect, this documents features a method for reducing the amount of circulating contrast agent within a mammal, wherein the mammal received a contrast agent containing a material capable of being attracted to a magnetic field. The method comprises, or consists essentially of: (a) obtaining a device comprising: (i) a guide catheter defining a lumen and having a proximal end and a distal end, wherein the guide catheter is configured to be inserted into a blood vessel of a mammal; and (ii) a capture element comprising: (1) an elongate member having a proximal end portion and a distal end portion, and (2) a magnetic field source located at or near the distal end portion, wherein the capture element is configured to be at least partially housed within the guide catheter, and wherein the magnetic field source is movable, relative to the guide catheter, in a direction away from the distal end of the guide catheter when the distal end of the guide catheter is located within the circulatory system of the mammal, (b) inserting the magnetic field source into the circulatory system, and (c) exposing blood of the mammal to a magnetic field from the magnetic field source under conditions wherein the contrast agent is attracted to the magnetic field and contacts the device, thereby reducing the amount of circulating contrast agent within the mammal. The mammal can be a human. The magnetic field source can be inserted into a right atrium, superior vena cava, inferior vena cava, coronary sinus, or pulmonary artery. The magnetic field can have a Gaussian strength of between 0.1 and 10,000 Gauss.

In another aspect, this documents features a method for performing a contrast agent imaging procedure having reduced risk of contrast agent-induced toxicity. The method comprises, or consists essentially of: (a) administering a contrast agent into a mammal, wherein the contrast agent comprises a material capable of being attracted to a magnetic field, (b) obtaining a device comprising: (i) a guide catheter defining a lumen and having a proximal end and a distal end, wherein the guide catheter is configured to be inserted into a blood vessel of the mammal; and (ii) a capture element comprising: (1) an elongate member having a proximal end portion and a distal end portion, and (2) a magnetic field source located at or near the distal end portion, wherein the capture element is configured to be at least partially housed within the guide catheter, and wherein the magnetic field source is movable, relative to the guide catheter, in a direction away from the distal end of the guide catheter when the distal end of the guide catheter is located within the circulatory system of the mammal, (c) obtaining an image from the mammal, (d) inserting the magnetic field source into the circulatory system, and (e) exposing blood of the mammal to a magnetic field from the magnetic field source under conditions wherein the contrast agent is attracted to the magnetic field and contacts the device, thereby reducing the amount of circulating contrast agent within the mammal. The mammal can be a human. The contrast agent can comprise iodine. The contrast agent can be injected into a coronary artery, systemic circulation, organ specific blood vessel, or a body cavity containing space. The image can be an X-ray image, MRI image, radioactive scan image, fluorescent image, PET image, or CT image. The magnetic field source can be inserted into a right atrium, superior vena cava, inferior vena cava, coronary sinus, or pulmonary artery. The magnetic field can have a Gaussian strength of between 0.1 and 10,000 Gauss. The inserting step can be performed before obtaining the image from the mammal. The inserting step can be performed after obtaining the image from the mammal. The inserting step can be performed before the administering step. The inserting step can be performed after the administering step.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a view of a device having a guide catheter with a capture element located within the guide catheter.

FIG. 2 is a view of the device depicted in FIG. 1 with the capture element extending outside the guide catheter.

FIG. 3 is a view of a device having a guide catheter with a capture element located within the guide catheter.

FIG. 4 is a view of the device depicted in FIG. 3 with the capture element extending outside the guide catheter.

FIG. 5 is a view of the device depicted in FIG. 3 with the capture element located within a tightly fitting guide catheter.

FIG. 6 is a view of the device depicted in FIG. 5 with an expanded view of the capture element extending outside a tightly fitting guide catheter.

FIG. 7 is a view of a device having a guide catheter with a capture element located within the guide catheter.

FIG. 8 is a view of the device depicted in FIG. 7 with the capture element extending outside the guide catheter.

FIG. 9 is a view of the device depicted in FIG. 7 located within a blood vessel with the capture element extending outside the guide catheter.

FIG. 10 is a view of a device having a guide catheter with a capture element located within the guide catheter.

FIG. 11 is a view of the device depicted in FIG. 10 with the capture element extending outside the guide catheter.

FIG. 12 is a view of the device depicted in FIG. 10 with an expanded view of the capture element located within a tightly fitting guide catheter.

### DETAILED DESCRIPTION

This document provides methods and materials that can be used to reduce or prevent contrast agent-induced toxicity. In general, the methods provided herein can include administering a contrast agent to a mammal and magnetically capturing at least some of the administered contrast agent so as to reduce the amount of contrast agent circulating within said mammal. For example, this document provides methods that can include administering a contrast agent containing a material capable of being attracted to a magnetic field. In some cases, a contrast agent can be an MRI contrast agent such as Gd-labeled albumin, Gd-labeled dextran, chromium-labeled red blood cells, gadolinium oxide, superparamagnetic iron oxide, ultrasmall superparamagnetic iron oxide (USPIO) particles, and hepatobiliary contrast agents, or an X-ray contrast agent. An X-ray contrast agent can contain iodine (e.g., iohexol, iodixanol, or ioversol) or barium. In some cases, hafnium, tantalum, tungsten, and oxides can be used as X-ray contrast agents. Examples of commercially available contrast agents include, without limitation, Myoview™ (technetium Tc-99m tetrofosmin), Omnipaque™ (iohexol), Optison™ (Perflutren Protein-Type A Microspheres for Injection, USP), Visipaque™ (iodixanol), Omniscan™ (gadodiamide), Visipaque™ (iodixanol), Omnipaque™ (iohexol), Omniscan™ (gadodiamide), and Visipaque™ (iodixanol). In some cases, the devices and methods provided herein can be adapted to capture agents other than or in addition to MRI or X-ray contrast agents. Such other agents include, without limitation, agents designed for use in Positron Emission Tomography (PET), radioactive imaging, fluorescent imaging, or other imaging techniques.

As described herein, a contrast agent can contain a material capable of being attracted to a magnetic field. For example, a contrast agent can be attached to a paramagnetic (e.g., magnesium, molybdenum, lithium, and tantalum), ferromagnetic (e.g., iron, nickel, and cobalt), or superparamagnetic material (e.g., a particle or nanoparticle). Any type of attachment can be used to attach a contrast agent and a material capable of being attracted to a magnetic field. For example, a contrast agent and paramagnetic, ferromagnetic, or superparamagnetic material can be chelated. Examples of contrast agents, contrast agents containing material capable of being attracted to a magnetic field, and methods for making such contrast agents are provided elsewhere (see, e.g., U.S. Pat. No. 5,324,503; U.S. Pat. No. 5,660,814; and U.S. Patent Application Publication No. 2005/0113675).

A contrast agent provided herein can be administered to any part of a mammal's body. For example, a contrast agent can be administered to a body cavity to be imaged, an organ to be imaged, a body part to be imaged, or the blood supply upstream of an organ or body part to be imaged. In some cases, a contrast agent can be administered intravenously, intraarterially, intrathecally, or intraabdominally. When imaging heart tissue or the cardiac region of a mammal, a contrast agent can be administered to a coronary artery via an intra-arterial injection. When imaging a specific organ or tissue in a mammal, a contrast agent can be administered to the target organ via a tissue or organ selective blood vessel.

After administering a contrast agent to a mammal, an image of the desired location can be obtained. For example, an MRI or X-ray image can be obtained after an MRI or X-ray contrast agent is administered to a human patient.

The devices provided herein can be used to remove a percentage (e.g., up to 5, 10, 15, 20, 25, 30, 35, 40, 45, 50, 55, 60, 65, 70, 75, 80, 85, 90, 95, or 100 percent) of the contrast agent administered to a mammal. In some cases, the devices provided herein can be used to remove between 5 and 75 percent of the contrast agent administered to a mammal. Such a removal can help reduce the mammal's risk of developing contrast agent-induced toxicity (e.g., contrast agent-induced nephropathy). Contrast agent-induced nephropathy as used herein with respect to a human refers to a condition where a human has either a greater than 25% increase of serum creatinine or an absolute increase in serum creatinine of 0.5 mg/dL.

In general, a device provided herein can contain a guide catheter and a capture element. A guide catheter can be configured to house a capture element and can be configured to be inserted into a blood vessel within a mammal (e.g., a mammal's femoral vein or artery). A capture element can be capable of supplying a magnetic field that can be positioned in the blood stream of a mammal. For example, a device provided herein can be configured to have the ability to position a magnetic field source of a capture element downstream of an organ or body part to be imaged such that a percentage of the administered contrast agent containing a material capable of being attracted to a magnetic field is captured before proceeding to other locations of the body (e.g., the mammal's kidneys). In some cases, a capture element can contain an elongate member and a magnetic field source. An elongate member can be a wire or catheter that is configured to fit within a guide catheter in a movable manner. In some cases, an elongate element can extend through the length of the guide catheter, and the magnetic field source can be positioned at a distal end of the elongate element. A user can manipulate a proximal end of a capture element to alter the location or position of a magnetic field source attached to a distal end portion of a capture element.

A guide catheter can be made of soft, pliable material (e.g., polytetrafluoroethylene (PTFE), polyethylene, polypropylene, or any other suitable material). An elongate member of a capture element can be made of a rigid or semi-rigid material (e.g., wire) or any other suitable material that can provide the elongate member with at least temporary rigidity so as to allow deployment or positioning of a magnetic field source. Examples of such materials include, without limitation, non-magnetized metals that can develop a magnetic field by induced magnetism via a strong magnetic field applied outside the body (e.g., MRI or Stereotaxis).

A magnetic field source can be a permanent magnet or an electromagnet. For example, a capture element can include an electromagnetic coil. A magnetic field source can provide a Gaussian strength of about 0.1 to about 10,000 Gauss (e.g., about 100 to about 10,000 Gauss). In some cases, a magnetic field source can be configured into a fiber network or a mesh. A fiber network can be a disordered array of magnetic fibers capable of supplying a magnetic field. Such a fiber network can define openings such that blood can flow through the fiber network. A mesh can be configured to define openings (e.g., an orderly array of openings) that allow blood can flow through the mesh. For example, a mesh can have a honeycomb-like structure. In some cases, a mesh containing a magnetic field source can be made of a shape memory material (e.g., nitinol). In such cases, the mesh can be configured to assume an expanded shape when the mesh exits the distal end of the guide catheter.

Any portion of a capture element (e.g., an elongate member, a magnetic field source, or both) can be covered on its exterior surface with a coating (e.g., PTFE, DACRON®, or other suitable material) to, for example, prevent tissue trauma. Examples of suitable coatings are described elsewhere (e.g., U.S. Patent Application Publication No. 2005/0277959). In some cases, a capture element can include an electromagnetic coil that contains a coating. Such a coating can form any shape. For example, a coating can be shaped to form pleats or fingers. In such cases, an increased surface area for contrast agent attachment can be provided.

In some cases, a capture element can include an expandable balloon structure that includes a magnetic field source. For example, a device provided herein can contain an expandable balloon at least partially surrounded with an expandable mesh capable of supplying a magnetic field. The balloon and mesh can be configured to assume an expanded shape when the balloon and mesh exit the distal end of the guide catheter and the balloon is inflated.

A device provided herein can include one or more additional elements to assist with positioning of a guide catheter or a capture element. These additional elements can be contained within the guide catheter or within an outer sheath that also contains the guide catheter. Positioning elements can be deflecting and/or steerable to, for example, facilitate their positioning within a device. Appendage positioning elements include, for example, suction catheters, forceps, and cryogenic-tipped catheters, which can be used to, for example, position a capture element. See, e.g., U.S. Patent Application Publication Nos. 2005/0154404 and 2004/0030335, and U.S. Pat. No. 6,488,689. In some cases, a device provided herein can be positioned by an externally applied magnetic field.

A device provided herein can include a removal element configured to remove captured items from the mammal in an intermittent or continuous manner. For example, a device provided herein can include a removal element that provides suction to a portion of a capture element that captures items (e.g., contrast agent). The suction can draw any captured items from the distal end of the device to a location outside a mammal's body. For example, a tube or lumen can be engineered into a shaft of a capture element such that agents captured by the magnetic field source can be removed intermittently or constantly without having to remove the device from a mammal's body. In such cases, a suction device can be attached to the end of the tube or lumen that is positioned outside a mammal's body during use.

The devices provided herein can have any suitable length and width (e.g., diameter). For example, a device can have a length between about 30.5 cm and about 183 cm (e.g., between about 61 cm and 153 cm, between about 76 cm and about 137 cm, or between about 91 cm and about 122 cm), such that its distal end can be placed within the cardiac region of a human and its proximal end can be positioned outside the human's body. Further, a device can have any suitable diameter. For example, a device can have an overall diameter (e.g., diameter of the outer sheath, or diameter of the guide catheter if there is no outer sheath) suitable for passage through the circulatory system and into a coronary sinus. In some cases, a device can have a diameter between about 0.05 cm and about 1.5 cm, between about 0.1 cm and about 1.0 cm, between about 0.15 cm and about 0.5 cm, between about 0.2 cm and about 0.4 cm, or about 0.2 cm, about 0.3 cm, or about 0.4 cm. In some cases, a device can have an unexpanded diameter between about 0.1 mm and about 1.5 cm, between about 0.5 mm and about 1.0 cm, or between about 1.0 mm and about 0.5 cm. Such an unexpanded diameter can be configured to expand to a maximum expanded diameter between 0.15 mm and 15 cm (e.g., between 0.15 mm and 5 mm).

A device can include any suitable mechanism to facilitating advancing an elongate element so that a magnetic field source extends away from the distal end of a guide catheter. For example, an elongate member (e.g., wire) of a capture element can be “cocked” with a spring mechanism. A clinician can actuate the spring mechanism, and the resulting forward pressure applied on the capture element can cause an elongate member to advance a pre-set distance. For example, the length of an elongate member that exits the guide catheter can be limited to between about 0.1 mm and about 15 cm.

The devices provided herein can be readily deployed in a percutaneous manner. In addition, the devices can be adapted to minimize trauma to the tissue they contact such that there is little or no erosion through the tissue, reducing the likelihood of bleeding and cardiac tamponade. Further, the devices can be reversible and/or repositionable, such that a clinician can position the capture element as desired.

In some cases, the devices and methods provided herein can be adapted to capture items other than or in addition to contrast agents. Such other items can include, without limitation, chemicals, drugs, toxins, immune complexes, pathogens, normal cells, malignant cells, and nano-devices. For example, a device provided herein can be designed to capture contrast agents and pathogens. In some cases, the devices and methods provided herein can be designed to use a magnetic field source, a binding force, an electrostatic force, or a combination thereof. For example, a device provided herein can be adapted to have a capture element having ligands or antibodies with the ability to bind a particular item (e.g., a toxin, immune complex, pathogen, normal cell, or malignant cell). In some cases, the devices and methods provided herein can be configured such that a drug attached to biotin can be administered to a mammal and then captured using a capture element coated with streptavidin. In some cases, the devices and methods provided herein can be configured such that a drug attached to streptavidin can be administered to a mammal and then captured using a capture element coated with biotin.

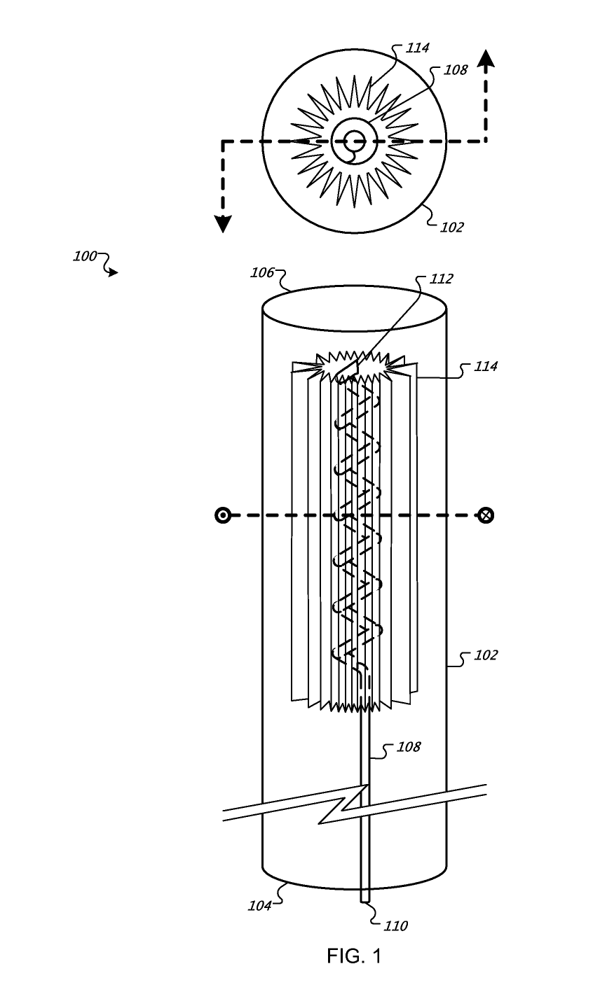

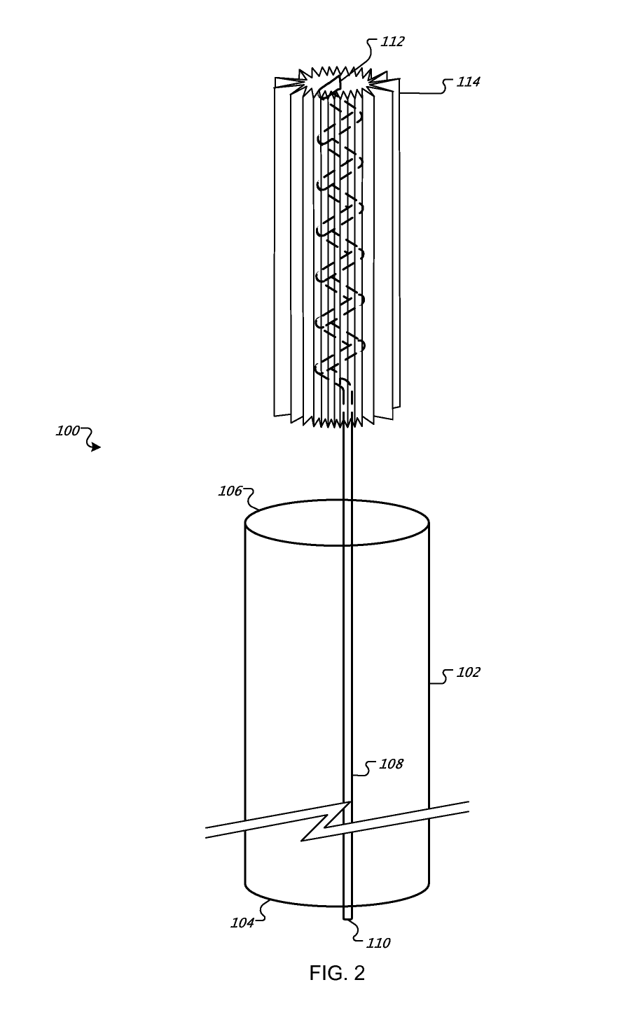

In reference to FIGS. 1 and 2, device 100 can contain guide catheter 102 having a proximal end 104 and a distal end 106. Guide catheter 102 can house at least a portion of capture element 108. Capture element 108 can have proximal end portion 110 and distal end portion 112. Proximal end portion 110 can be configure to form an elongate member. A part of distal end portion 112 can be configured to form a magnetic field source. For example, FIGS. 1 and 2 depict the magnetic field source as an electromagnetic coil, which can be powered via a battery or external power source. A magnetic field source can be surrounded by a coating 114 of any shape. In FIGS. 1 and 2, coating 114 is shaped to provide pleats, which can increase the surface area available to capture contrast agent. FIG. 1 depicts device 100 in a configuration where distal end portion 112 is located within guide catheter 102, while FIG. 2 depicts device 100 in a configuration where distal end portion 112 is located outside guide catheter 102. The configuration depicted in FIG. 1 can be used while inserting device 100 into a mammal or withdrawing device 100 from a mammal. The configuration depicted in FIG. 2 can be used to capture contrast agent from blood within a mammal.

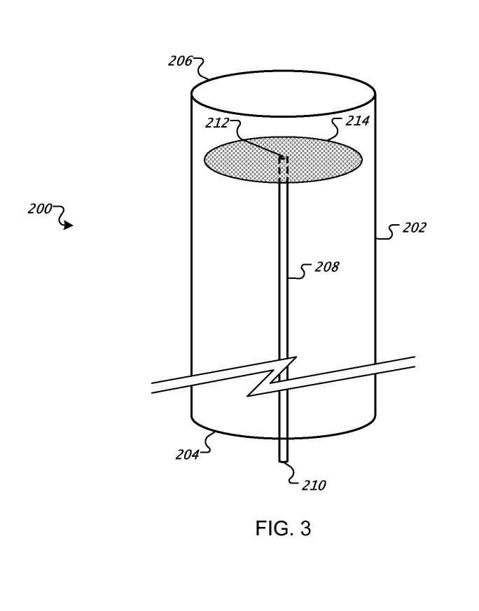

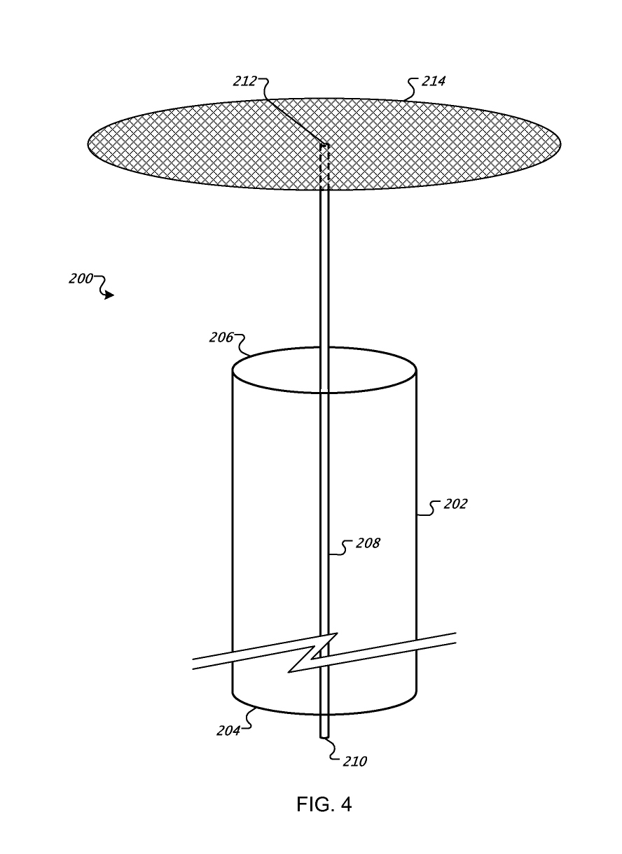

In reference to FIGS. 3 and 4, device 200 can contain guide catheter 202 having a proximal end 204 and a distal end 206. Guide catheter 202 can house at least a portion of capture element 208. Capture element 208 can have proximal end portion 210 and distal end portion 212. Proximal end portion 210 can be configured to form an elongate member. A part of distal end portion 212 can be configured to form a magnetic field source 214. For example, FIGS. 3 and 4 depict magnetic field source 214 as a expandable mesh. As indicated herein, a magnetic field source can be a permanent magnet or an electromagnet such as an electromagnet powered via a battery or external power source. FIG. 3 depicts device 200 in a configuration where distal end portion 212 is located within guide catheter 202, while FIG. 4 depicts device 200 in a configuration where distal end portion 212 is located outside guide catheter 202. The configuration depicted in FIG. 3 can be used while inserting device 200 into a mammal or withdrawing device 200 from a mammal. The configuration depicted in FIG. 4 can be used to capture contrast agent from blood within a mammal. The mesh can be shaped and configured in any three dimensional structure. For example, a mesh can have a plurality of layers, uniform or variable mesh sizes, multiple magnetic components, or one or more protective layers.

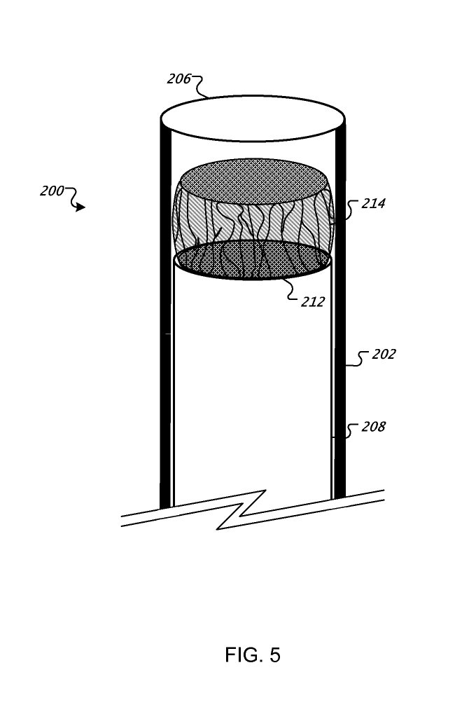

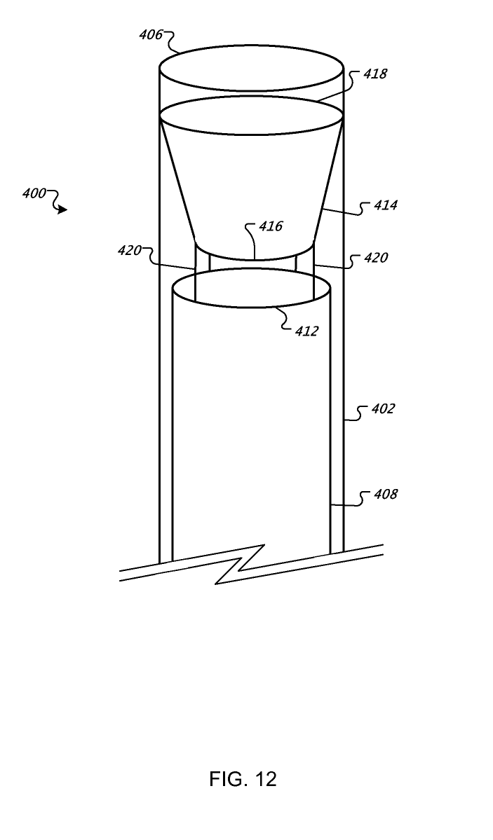

In some embodiments, the elongated portion of a capture element can fit closely within a guide catheter. For example, a capture element can be located within a tightly fitting guide catheter as depicted in FIGS. 5, 6, and 12. With reference to FIG. 5, magnetic field source 214 is in the form of a mesh that can be deformed to fit within guide catheter 202 when capture element 208 is completely retracted into guide catheter 202. As explained above, magnetic field source 214 can expand when the distal end portion 212 of capture element 208 is extended outside guide catheter 202. See, e.g.,

FIG. 4.

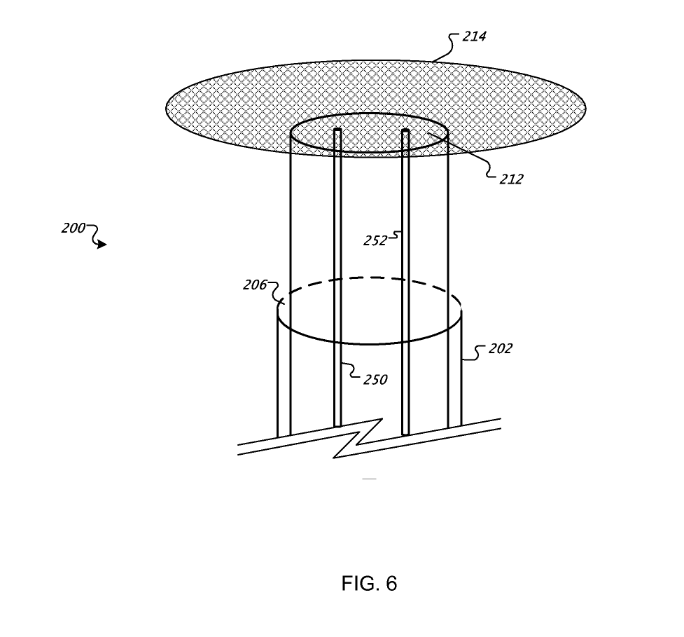

As indicated herein, a magnetic field source can be powered via a battery or external power source. For example, electrical leads can extend from a battery or external power source to a magnetic field source. With reference to FIG. 6, electrical leads 250 and 252 can extend from magnetic field source 214 to a battery or external power source.

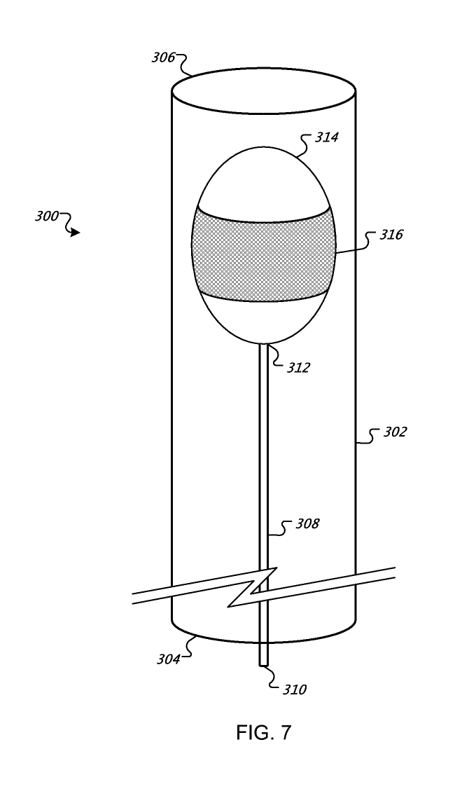

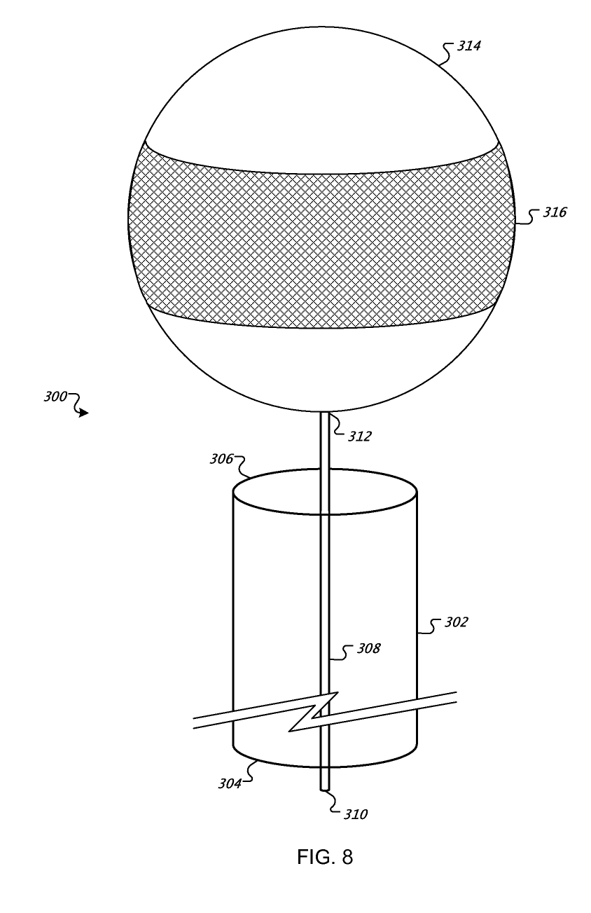

In reference to FIGS. 7 and 8, device 300 can contain guide catheter 302 having a proximal end 304 and a distal end 306. Guide catheter 302 can house at least a portion of capture element 308. Capture element 308 can have proximal end portion 310 and distal end portion 312. Proximal end portion 310 can be configure to form an elongate member. A part of distal end portion 312 can be attached to expandable balloon 314 having magnetic field source 316 in the form of an expandable mesh. FIG. 7 depicts device 300 in a configuration where distal end portion 312 is located within guide catheter 302, while FIG. 8 depicts device 300 in a configuration where distal end portion 312 is located outside guide catheter 302. The configuration depicted in FIG. 7 can be used while inserting device 300 into a mammal or withdrawing device 300 from a mammal. The configuration depicted in FIG. 8 can be used to capture contrast agent from blood within a mammal.

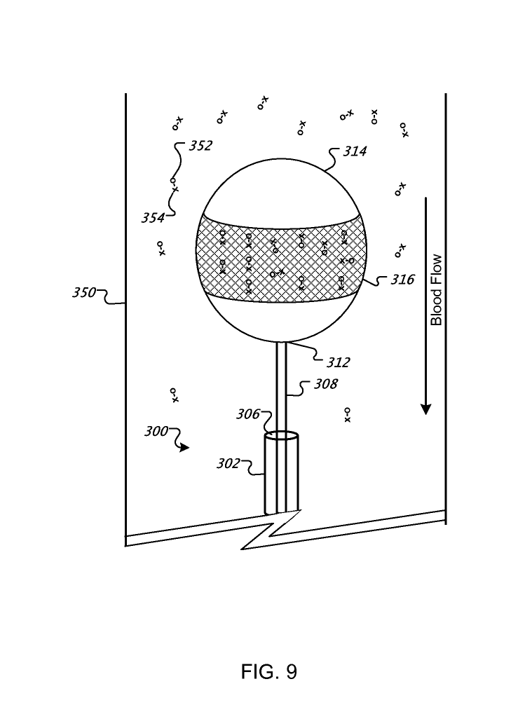

As described herein, a device provided herein can be placed into a blood vessel to capture agents from blood. FIG. 9 depicts an example of the use of the devices provided herein. In this case, device 300 can extend from outside a mammal into blood vessel 350 of a mammal. The devices can extend into a blood vessel in either direction: upstream against blood flow as shown in FIG. 9 or down stream with blood flow. As depicted in FIG. 9, guide catheter 302 can be placed within blood vessel 350. Once in position, capture element 308 can be deployed such that capture element 308 extends beyond distal end 306. In this example, expandable balloon 314 can be inflated, and agent 352 coupled to material 354, which is capable of being attracted to a magnetic field, can be captured by magnetic field source 316. During use of the devices provided herein, the concentration of agents upstream of the device can be higher than the concentration of agents downstream of the device.

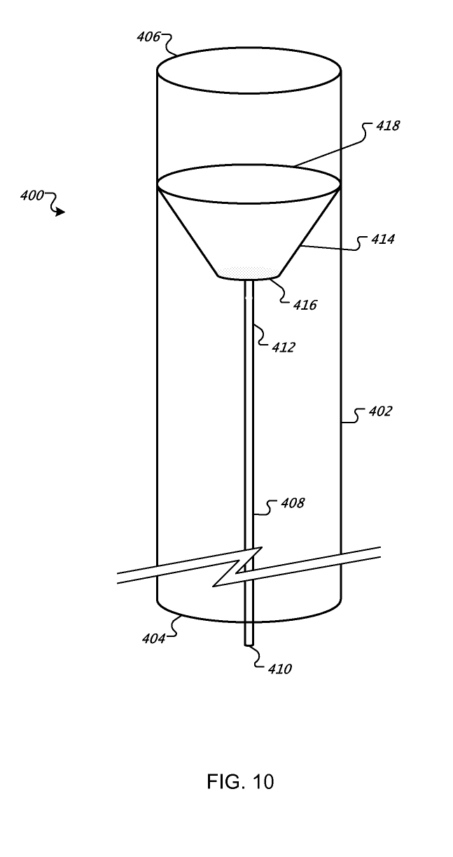

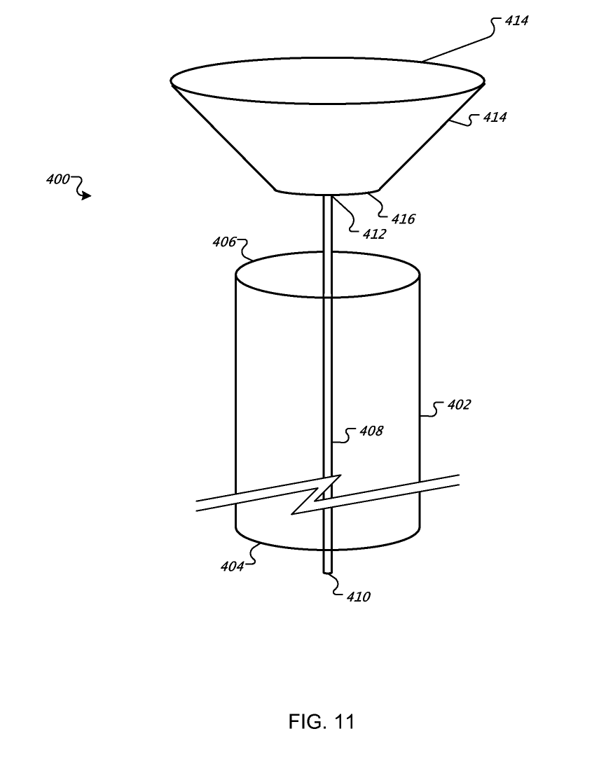

In reference to FIGS. 10 and 11, device 400 can contain guide catheter 402 having a proximal end 404 and a distal end 406. Guide catheter 402 can house at least a portion of capture element 408. Capture element 408 can have proximal end portion 410 and distal end portion 412. Proximal end portion 410 can be configure to form an elongate member. A part of distal end portion 412 can be configured to form a magnetic field source. For example, a part of distal end portion 412 can be configured to form funnel 414 having inlet opening 418 and outlet opening 416. All or a portion of the inner surface of funnel 414 can form a magnetic field source. For example, the entire inner surface of funnel 414 can contain magnetic material, thereby forming a magnetic field source. In some cases, outlet opening 416 can contain a mesh that forms a magnetic field source. For example, outlet opening 416 can be covered with a mesh that forms a magnetic field source. In some cases, the inner surface of funnel 414 can be pleated to increase the surface area of the inner surface.

FIG. 10 depicts device 400 in a configuration where distal end portion 412 is located within guide catheter 402, while FIG. 11 depicts device 400 in a configuration where distal end portion 412 is located outside guide catheter 402. The configuration depicted in FIG. 10 can be used while inserting device 400 into a mammal or withdrawing device 400 from a mammal. The configuration depicted in FIG. 11 can be used to capture contrast agent from blood within a mammal.

In some embodiments, the elongated portion of a capture element can fit closely within a guide catheter. For example, a capture element can be located within a tightly fitting guide catheter as depicted in FIGS. 5, 6, and 12. With reference to FIG. 5, magnetic field source 214 is in the form of a mesh that can be deformed to fit within guide catheter 202 when capture element 208 is completely retracted into guide catheter 202. As explained above, magnetic field source 214 can expand when the distal end portion 212 of capture element 208 is extended outside guide catheter 202. See, e.g., FIG. 4.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A device comprising:
(a) a catheter configured to be inserted into a blood vessel of a mammal; and
(b) a capture element comprising:
(i) an elongate member, and
(ii) a magnetic field source,
wherein said capture element is configured to be at least partially housed within said guide catheter, and
wherein said magnetic field source is movable, relative to said guide catheter.

2. The device of claim 1, wherein said elongate member comprises a proximal end portion and a distal end portion, and wherein said magnetic field source is located at or near said distal end portion.

3. The device of claim 1, wherein said magnetic field source is movable, relative to said guide catheter, in a direction away from a distal end of said guide catheter when said distal end of said guide catheter is located within the circulatory system of said mammal.

4. The device of claim 1, wherein said mammal is a human.

5. The device of claim 1, wherein said guide catheter is between 10 cm and 125 cm in length.

6. The device of claim 1, wherein said guide catheter is between 0.1 mm and 0.4 cm in diameter.

7. The device of claim 1, wherein said elongate member comprises a wire.

8. The device of claim 1, wherein said magnetic field source comprises an electromagnet.

9. The device of claim 1, wherein said capture element comprises a coating located at least partially around said magnetic field source.

10. The device of claim 9, wherein said coating comprises an inner surface and an outer surface, wherein said outer surface is configured to contact a contrast agent containing a material capable of being attracted to a magnetic field when a distal end of said guide catheter is located within the circulatory system of said mammal and said mammal received said contrast agent.

11. The device of claim 10, wherein said coating comprises projections that extend away from said magnetic field source, thereby increasing the surface area of said coating for contact with said contrast agent.

12. The device of claim 9, wherein said coating is a pleated coating.

13. The device of claim 1, wherein said capture element comprises a fiber network located at or near a distal end portion of said elongate member, wherein said fiber network comprises said magnetic field source.

14. The device of claim 13, wherein said fiber network defines spaces having a diameter capable of allowing blood flow through said fiber network.

15. The device of claim 1, wherein said capture element comprises a mesh located at or near a distal end portion of said elongate member, wherein said mesh comprises said magnetic field source.

16. The device of claim 15, wherein said mesh defines spaces having a diameter capable of allowing blood flow through said mesh.

17. The device of claim 15, wherein said mesh is an expandable mesh.

18. The device of claim 1, wherein said capture element comprises an expandable balloon attached to an expandable mesh.

19. A method for reducing the amount of circulating agent within a mammal, wherein said mammal received an agent containing a material capable of being attracted to a magnetic field, said method comprising:
(a) inserting a device comprising a magnetic field source into a blood vessel of said mammal, and
(b) exposing blood of said mammal to a magnetic field from said magnetic field source under conditions wherein said agent is attracted to said magnetic field and contacts said device, thereby reducing the amount of circulating agent within said mammal.

20. A method for reducing the amount of circulating contrast agent within a mammal, wherein said mammal received a contrast agent containing a material capable of being attracted to a magnetic field, said method comprising:
(a) obtaining a device comprising:
(i) a catheter configured to be inserted into a blood vessel of a mammal; and
(ii) a capture element comprising:
(1) an elongate member, and
(2) a magnetic field source,
wherein said capture element is configured to be at least partially housed within said guide catheter, and
wherein said magnetic field source is movable, relative to said guide catheter,
(b) inserting said magnetic field source into said circulatory system, and
(c) exposing blood of said mammal to a magnetic field from said magnetic field source under conditions wherein said contrast agent is attracted to said magnetic field and contacts said device, thereby reducing the amount of circulating contrast agent within said mammal.

21. A method for performing a contrast agent imaging procedure having reduced risk of contrast agent-induced toxicity, wherein said method comprises:
(a) administering a contrast agent into a mammal, wherein said contrast agent comprises a material capable of being attracted to a magnetic field,
(b) obtaining a device comprising:
(i) a catheter configured to be inserted into a blood vessel of a mammal; and
(ii) a capture element comprising:
(1) an elongate member, and
(2) a magnetic field source,
wherein said capture element is configured to be at least partially housed within said guide catheter, and
wherein said magnetic field source is movable, relative to said guide catheter,
(c) obtaining an image from said mammal,
(d) inserting said magnetic field source into said circulatory system, and
(e) exposing blood of said mammal to a magnetic field from said magnetic field source under conditions wherein said contrast agent is attracted to said magnetic field and contacts said device, thereby reducing the amount of circulating contrast agent within said mammal.
